(19) United States
(12) Patent Application Publication
(10) Pub. No.: US 2013/0275155 A1
(43) Pub. Date: Oct. 17, 2013

(54) PULSE OXIMETERS AND SYSTEMS AND METHODS FOR OBTAINING DATA THEREFROM

(71) Applicant: Kyle William Miko, Coral Springs, FL (US)

(72) Inventor: Kyle William Miko, Coral Springs, FL (US)

(21) Appl. No.: 13/862,356

(22) Filed: Apr. 12, 2013

Related U.S. Application Data
(60) Provisional application No. 61/623,535, filed on Apr. 12, 2012.
(60) Provisional application No. 61/703,240, filed on Sep. 19, 2012.

(51) Int. Cl. A61B 5/00; G06Q 50/24; G06F 19/00

(52) U.S. Cl. CPC A61B 5/0004; G06F 19/36; G06Q 50/24
USPC 705/3; 600/323

(57) ABSTRACT

A tele-health solution promotes patient wellness and reduces healthcare costs while improving patient compliance. To qualify for home oxygen or CPAP machines, a user must monitor their blood oxygen saturation (SpO₂), typically overnight. A DME provider is often used to provide the monitoring equipment, retrieve the monitoring equipment and get the data obtained to the physician. A system is discloses that allow the data obtained to be sent wirelessly, for example, via general packet radio service or the like, to a computer server that can generate the necessary report and electronic submit such report to the physician. This process can take minutes, rather than days. Moreover, the pulse oximeter can be outfitted with a good study indicator which monitors the time that data has been successfully measured by the device, allowing a determination of whether the data is adequate or if the device needs to be worn for another night.

### CROSS-REFERENCE TO RELATED APPLICATIONS

The present invention claims the benefit of priority of U.S. provisional patent application No. 61/623,535, filed Apr. 12, 2012, and U.S. provisional patent application 61/703,240, filed Sep. 19, 2012, the contents of both are herein incorporated by reference.

### BACKGROUND OF THE INVENTION

The present invention relates to pulse oximeters and, more particularly, to a wireless overnight oximeter providing a streamlined process for oxygen qualification via a wireless service. The present invention further related to a pulse oximeter that will display the amount of time a patient has used the device.

Pulse oximeters are often used in the medical field to determine a patient's saturation of peripheral oxygen (SpO₂). These measurements may be useful in determining if the patient is getting sufficient oxygen exchange through their breathing efforts. Medical conditions, such as chronic obstructive pulmonary disease (COPD), sleep apnea, or the like, may temporarily lower a patient's SpO₂ during their sleep. Durable medical equipment (DME) companies typically use overnight oximeters to determine if a patient may qualify for home oxygen under Medicare guidelines.

Currently, a durable medical equipment (DME) provider must deliver an overnight pulse oximeter to a patient's house. The next day, the DME provider must return to the patient's house to retrieve the device. The DME provider brings the device to their office where the device is uploaded to an independent diagnostic testing facility (IDTF). The results are then forwarded from the IDTF to the physician and/or DME provider. If the patient qualifies, the physician orders oxygen and the DME provider then delivers the oxygen to the patient.

The above process requires considerable amount of legwork on the part of the DME provider. This not only costs the DME provider in their time, but also potentially slows the qualification of the patient for home oxygen.

Many times, the patient does not wear the device long enough or at all, so the DME provider must initiate the entire process again. Current models of overnight oximeters do not display the amount of time the patient used the device. When the DME downloads the device data, they commonly find there is not enough data (typically two hours or more) to produce a report. The DME will then have to re-deliver the device to the patient and hope the patient wears the device enough time for the second data collection attempt.

As can be seen, there is a need for an improved apparatus and process for obtaining oximetry data from a patient. Moreover, there is a need for an overnight pulse oximeter that can track and provide a visual time indicator showing the amount of time a patient has used the device.

### SUMMARY OF THE INVENTION

In one aspect of the present invention, a method for obtaining diagnostic medical information from a patient comprises delivering a medical device to a patient to obtain medical data therefrom; sending the medical data wirelessly from the medical device to a centralized data server, where the medical device periodically sends the medical data and the centralized data server compiles the medical data received; generating a report from medical data received; and electronically transmitting the report to a physician.

In another aspect of the present invention, a pulse oximeter comprises a display specifying whether a sufficient amount of medical data has been collected by the pulse oximeter.

In a further aspect of the present invention, a method for overnight testing of a patient's blood oxygen concentration comprises distributing an overnight pulse oximeter to a patient; collecting pulse oximetry data overnight; reading a display on the overnight pulse oximeter, the display specifying whether a sufficient amount of data has been recorded on the overnight pulse oximeter; and retrieving the overnight pulse oximeter from the patient if the sufficient amount of data has been recorded, as shown on the display.

These and other features, aspects and advantages of the present invention will become better understood with reference to the following drawings, description and claims.

### BRIEF DESCRIPTION OF THE DRAWINGS

FIG. 1 is a schematic representation of a process for obtaining wireless pulse oximetry data from a patient according to an exemplary embodiment of the present invention; and

FIG. 2 is a front view of an overnight pulse oximeter having a good study indicator for measuring time of data acquisition, according to an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an innovative tele-health solution that promotes patient wellness and reduces healthcare costs while improving patient compliance. Many conditions are monitored at home, where the data later needs to be transmitted to a physician. Common conditions include irregular heartbeats or cardiac arrhythmias, blood pressure, glucose levels, lipid levels, and the like. To qualify for home oxygen or CPAP machines, for example, a user must monitor their blood oxygen saturation (SpO₂), typically overnight. A DME provider is often used to provide the monitoring equipment, retrieve the monitoring equipment and get the data obtained to the physician. The system of the present invention allows the data obtained to be sent wirelessly, for example, via general packet radio service (GPRS) or the like, to a computer server that can generate the necessary report and electronic submit such report to the physician. This process can take minutes, rather than days. Moreover, the pulse oximeter can be outfitted with a good study indicator which monitors the time that data has been successfully measured by the device, allowing a determination of whether the data is adequate or if the device needs to be worn for another night.

In some embodiments, an overnight pulse oximeter can be designed to be sent back to the DME provider rather than to transmit the information wirelessly. This type of device may be helpful where, for example, a wireless signal may not be available or where conventional delivery and return methods are desired. The overnight pulse oximeter can display the amount of time data has been recorded. With this display being present, a DME provider can simply call the patient and ask the patient to verbally confirm the amount of time the patient wore the device. Now, the DME provider can absolutely know if the patient wore the device long enough to produce a report before driving out to the patient's home to collect the device from the patient.

If the data obtained happens to be insufficient, In some embodiments, the device will include an indicator (a good study indicator, for example, showing the time that data has been recorded, as discussed above) that the device needs to be worn another night. In some embodiments, the data can be wirelessly sent to the DME provider and the DME provider can simply ask the patient to wear the device again and wirelessly obtain the additional data thereafter.

The system of the present invention allows DME providers to operate with greater efficiency and thereby save activity-based costs in the oxygen qualification process conventionally deployed. The system of the present invention can utilize existing cell phone technology to transmit overnight data in real-time and eliminate costly retrieval and upload process steps that help save money. Of course, other wireless data communication methods are contemplated within the scope of the present invention.

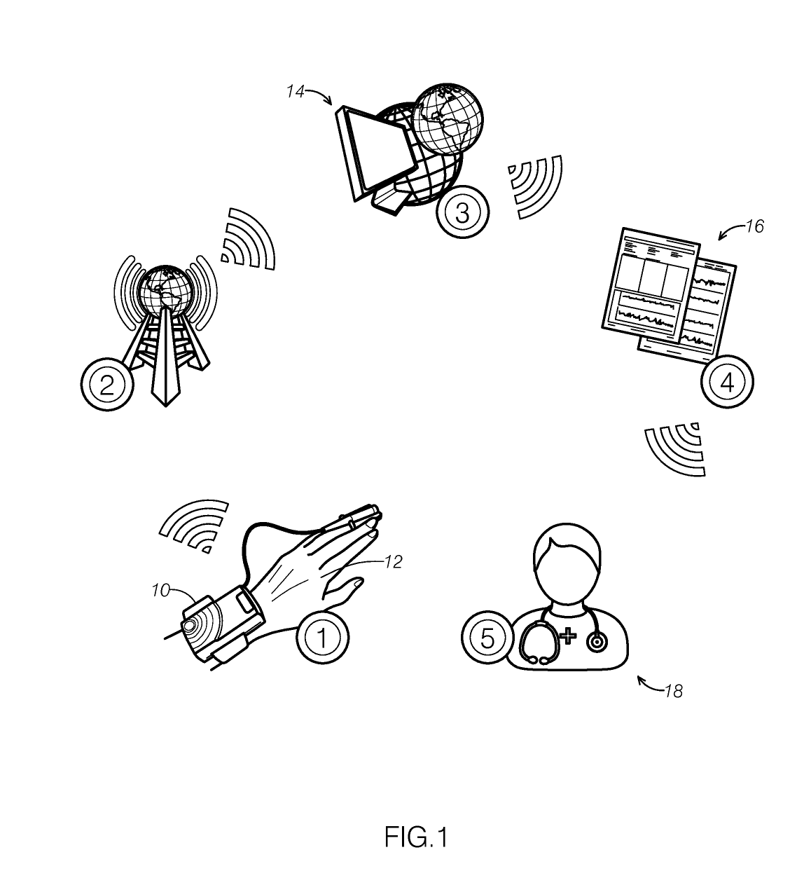

Referring to FIG. 1, the system can include a wireless pulse oximetry device 10 worn by the patient 12. The device 10 can include data storage and transmission features. For example, up to about 72 hours or more of data storage can be available within the pulse oximetry device 10. The device 10 can be powered by a rechargeable battery (not shown) so that the device 10 can be easily charged and delivered to the patient 12 when needed.

The device 10 can be configured to automatically communicate with a data server 14, such as a cloud-based data storage device. In some embodiments, the device 10 can periodically communicate with the data server 14. For example, every 15 minutes, the device 10 can automatically transmit data to the data server 14. The data server 14 can include software for generating an appropriate report 16, depending on the data being monitored and its purpose. The report 16 can be electronically transmitted to a physician 18 within minutes of the test completion.

In the example of home oxygen qualification, if the patient qualifies, the physician orders the oxygen and the DME provider delivers the oxygen to the patient. The DME provider can pick up the wireless pulse oximetry device 10 when making the oxygen delivery, thus saving extra trips to the patient's location. Under conventional methods, the DME provider would have to return to pick up the device and then return again to make the oxygen delivery (along with two or more additional trips if insufficient data has been collected and the device has to be re-delivered and re-retrieved).

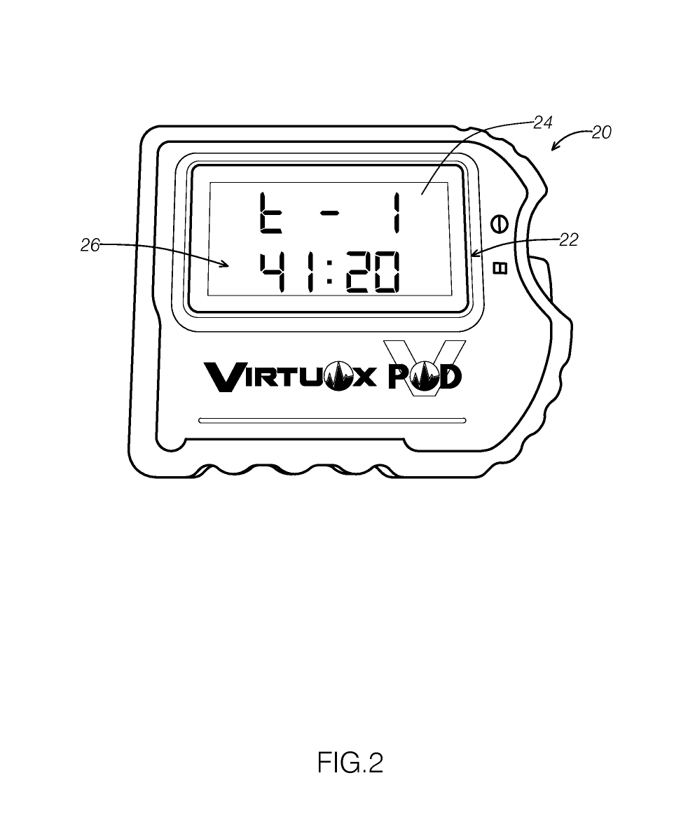

Referring now to FIG. 2, a pulse oximeter device 20 may include a screen 22 that can indicate an amount of time 26 data has been collected. For example, the pulse oximeter device 20 may be disposed on a patient's wrist with a finger probe attached to the patient's finger. If, at some point during the night, the patient removes the finger probe, conventional pulse oximeter recording devices would need to be sent back to the DME company to download the data before they can know if sufficient data has been collected. However, with the pulse oximeter device 20 of the present invention, the patient can simply view the data recording time 26 on a screen on the device and determine if such an amount of data measurement is sufficient for the DME company's purposes. In some embodiments, a textual representation 24 may be present on the screen. The textual representation may include various displays, such as “time”, “total storage time”, “data collection time”, “sufficient data collected”, “insufficient data collected” or the like.

In some embodiments, the DME company can simply pre-program the device with the minimum data recording time so that, instead of displaying a time, the device can simply display an indicator 24, for example, a green light, to show that sufficient data has been gathered. In some embodiments, the data recording time 26 may flash, change colors, or the like to indicate sufficient data has been recorded.

The pulse oximeter devices 10, 20 may be made in various designs and may use various probes. For example, the probe may be a finger probe directly wired to the pulse oximeter itself. In some embodiments, the probe may be a wireless finger probe, sending a wireless signal, such as a Bluetooth signal, to a separate pulse oximeter recording device. Moreover, the pulse oximeter device 10, 20 may include various means for recording, saving, and accessing the data measurements. Regardless of configuration, probe type, and the like, the present invention relates to such pulse oximetry devices that include a time recording/display feature to indicate when sufficient data collection time has been achieved.

While the above description focuses on using a pulse oximeter for obtaining SpO₂ data from a patient, the system of the present invention can be used with other devices for obtaining and wirelessly transmitting other medical data.

It should be understood, of course, that the foregoing relates to exemplary embodiments of the invention and that modifications may be made without departing from the spirit and scope of the invention as set forth in the following claims.

What is claimed is:

1. A method for obtaining diagnostic medical information from a patient, comprising:
delivering a medical device to a patient to obtain medical data therefrom;
sending the medical data wirelessly from the medical device to a centralized data server, where the medical device periodically sends the medical data and the centralized data server compiles the medical data received;
generating a report from medical data received; and
electronically transmitting the report to a physician.

2. The method of claim 1, wherein the medical device is a pulse oximeter device.

3. The method of claim 1, further comprising determining whether the medical data has been obtained for a sufficient period of time by viewing a screen on the medical device.

4. The method of claim 3, wherein the screen shows a time over which the medical data was successfully collected by the medical device.

5. The method of claim 3, wherein the screen shows an indicator showing whether a sufficient amount of the medical data was successfully collected by the medical device.

6. A pulse oximeter comprising a display specifying whether a sufficient amount of medical data has been collected by the pulse oximeter.

7. The pulse oximeter of claim 6, wherein the display shows an amount of time that the pulse oximeter has collected data.

8. The pulse oximeter of claim 6, wherein the display shows an indicator when the sufficient amount of medical data is collected by the pulse oximeter.

9. A method for overnight testing of a patient's blood oxygen concentration, comprising:
distributing an overnight pulse oximeter to a patient;
collecting pulse oximetry data overnight;
providing a display on the overnight pulse oximeter, the display specifying whether a sufficient amount of data has been recorded on the overnight pulse oximeter; and
retrieving the overnight pulse oximeter from the patient if the sufficient amount of data has been recorded, as shown on the display.

10. The method of claim 9, wherein the display shows an amount of time over which data is recorded.

11. The method of claim 9, further comprising sending the data wirelessly from the overnight pulse oximeter to a centralized data server, where the overnight pulse oximeter periodically sends the medical data and the centralized data server compiles the data received.
